(19) United States
(12) Patent Application Publication
(10) Pub. No.: US 2025/0244323 A1
(43) Pub. Date: Jul. 31, 2025

(54) REAGENT ANALYZER SYSTEM AND METHOD FOR REAGENT STRIP AUTHENTICATION

(71) Applicant: Siemens Healthcare Diagnostics Inc., Tarrytown, NY (US)

(72) Inventors: Manish Deshpande, Newton, MA (US); Emma Orton, Cranston, RI (US)

(73) Assignee: Siemens Healthcare Diagnostics Inc., Tarrytown, NY (US)

(21) Appl. No.: 18/699,623

(22) PCT Filed: Oct. 13, 2022
(86) PCT No.: PCT/US2022/078003

§ 371 Date: Apr. 9, 2024

Related U.S. Application Data
(60) Provisional application No. 63/255,601, filed on Oct. 14, 2021.

(51) Int. Cl. G01N 33/543; G06F 21/44; G06K 7/12

(52) U.S. Cl. CPC G01N 33/54391; G06F 21/44; G06K 7/12

(57) ABSTRACT

A reagent strip is described. The reagent strip includes a substrate and at least one reagent pad positioned on the substrate. The substrate has a storage unit storing control information, such as an authentication code, encoded as a part of a non-visible code. The authentication code may be used to determine if the reagent strip is authentic before testing is done or results are produced.

This application claims benefit under 35 USC § 119 (e) of U.S. Provisional Application No. 63/255,601, filed Oct. 14, 2021. The entire contents of the above-referenced patent application are hereby expressly incorporated herein by reference.

### BACKGROUND

Early arcade games were easily copied due to lack of encryption. Instead, those early games usually relied on structural protections, such as memory that could be wiped when tampered with. However, these structural protections could be circumvented by various work arounds. Encryption was later adopted and often was only broken by information being lost or leaked.

When downloads and CDs became ubiquitous, encryption/keys were often the default method of protection. These methods of protection were often broken within weeks to months of a product's release-during the highest rate of consumption. Currently, the means of anti-piracy often utilized to ensure long term product protection is a superficial encryption coupled with a deeper protection.

For example, one of the Spyro™ games released for the Playstation 1™ console was encrypted such that the game file would only run/open on the Playstation™ system. Copying the game should have been as simple as burning a new CD with a copy of the original file. There was, however, a specific indexing system included on the original disk. Although, the game could be easily copied to a new CD and played initially, the inclusion of this specific indexing system meant that, as the game was played, slowly, certain parts would stop working, making the game functionally unplayable.

With respect to consumable products, many traditional authentication systems rely on processes that provide immediate detection of non-authentic products, such as the provision of a watermark in bank notes, or holograms in passports. Traditional authentication systems also rely on authentication systems that would impose great difficulty on anyone other than the manufacturer to perform or duplicate, such as processes that require the expense of specialized equipment or those that require a high degree of technical know-how. However, counterfeiters have become increasingly more sophisticated in undermining many traditional authentication systems.

Manufacturers employing an authentication system often do not attempt to hide the fact that their products include an authentication system. Unfortunately, however, these obvious and publicly known authentication systems simply alert possible counterfeiters to the systems that must be subverted to successfully counterfeit a given product. It is, then, usually only a matter of time before counterfeiters are able to undermine publicly known authentication systems. As such, manufacturers face ever increasing costs in implementing new or different authentication systems in an attempt to outpace the work of counterfeiters. This is particularly true among manufacturers of consumables, where it is critical that the end user has confidence that the product is not counterfeit.

In the field of reagent analyzers, for example, where the integrity of consumable products is of the utmost importance, consumable products known as reagent strips are used in the analysis of a variety of substances such as biological fluids and tissues, and commercial or industrial fluids. Reagent strips are affixed with a particular chemical that can indicate the presence of specific particulates within the substances that it is contacted with, when it is analyzed, either manually or instrumentally using a reagent analyzer.

Reagent strips enjoy wide use in many analytical applications, especially in the chemical analysis of biological fluids, because of their relatively low cost, ease of usability, and speed in obtaining results. In medicine, for example, numerous physiological functions can be monitored and/or diagnosed using reagent strips and a reagent analyzer. Such reagent strips provide physicians and laboratory technicians with a facile diagnostic tool.

Because of their widespread use, counterfeit reagent strips have hit the market. However, the performance of counterfeit reagent strips cannot be guaranteed and false readings may be obtained as a result of their use. Such false readings can have drastic implications with respect to the course of treatment a patient may receive.

Currently, manufacturers of reagent strips utilize instantly detectable authentication systems. These instantly detectable authentication systems include a security feature, such as infrared bands on a substrate of the reagent strips, described in U.S. Pat. No. 8,422,126. When a particular reagent strip is used and inserted into an analyzer, the security feature is read and a determination is made whether or not the reagent strip is authentic or counterfeit prior to reporting any results from reading a reagent pad on the reagent strip. If the reagent strip is determined to be counterfeit, then the results from reading the reagent pad are not reported to the user. However, such authentication systems can be counterfeit by copying the chemistry and/or IR band. The counterfeit products can also be tested by inserting the counterfeit reagent strip into a reagent analyzer and receiving an immediate determination of whether or not the reagent analyzer detected the counterfeit reagent strip.

Therefore, a need exists for a system and method of making and authenticating a consumable product that does not alert potential counterfeiters on how to counterfeit the consumable product. In addition, other control information such as, lot number, calibration information, manufacture date, and expiration date, for instance, that improve the accuracy of testing may be included. It is to such consumable products, systems, and methods that the inventive concepts disclosed herein are directed.

### SUMMARY

Methods and apparatuses to authenticate consumable products such as reagent strips are disclosed. According to some possible implementations, a reagent strip system may include a reagent strip having a storage unit and a non-visible code encoded in the storage unit, the non-visible code encoded with a substance that produces a signal, e.g., reflectance or absorption of light, when illuminated by an optical signal in a particular and known spectrum. For example, the substance can produce a visible light when illuminated by light in a non-visible spectrum. The reagent strip system may include an analyzer comprising an illumination device configured to project an optical signal in the particular and known spectrum onto at least a portion of the reagent strip, a reader configured to capture an image of the light produced by the non-visible code encoded in the storage unit of the reagent strip when illuminated by the optical signal from the illumination device, and a controller including a processor, and a non-transitory computer readable medium storing instructions. When the instructions are executed by the processor, the instructions cause the processor to extract control information from the non-visible code in the image of the light produced by the non-visible code encoded in the storage unit, and perform a predetermined action based upon the control information.

In some embodiments, the control information of the reagent strip system may further comprise a unique identifier value.

In some embodiments, the non-visible code of the reagent strip system may include one or more of: a 1D barcode, a 2D barcode, a QR code, a multi-dot pattern, and a numeric string.

In some embodiments, the analyzer of the reagent strip system may be a reagent strip analyzer.

In some embodiments, the analyzer of the reagent strip system may further comprise an input device, an output device, a communication device, or a combination thereof.

In some embodiments, the reader of the reagent strip system may be an imager.

In some embodiments, the non-visible code of the reagent strip system may comprise calibration information.

In some embodiments, the non-visible code of the reagent strip system may further comprise an expiration date of the reagent strip.

In some embodiments, the predetermined action causes the processor to output a notification to an output device, restrict further operation of the analyzer, or a combination thereof.

In some embodiments, the non-visible code may include a multi-dot pattern scheme and the non-transitory computer readable medium further stores a mapping scheme that the processor uses to decode the non-visible code to extract the control information.

According to some possible implementations, a reagent strip analyzer, may include an illumination device configured to project an optical signal in a non-visible spectrum. A reader may be included, the reader operable to read a non-visible code from each of a plurality of reagent strips when illuminated by the optical signal from the illumination device, each of the plurality of reagent strips having a storage unit storing the non-visible code, the non-visible code encoded with a substance only visible when illuminated by the optical signal in the non-visible spectrum. A controller may be included, the controller including a processor and a non-transitory computer readable medium storing instructions that, when executed by the processor, cause the processor to store the information in the non-transitory computer readable medium, analyze the information to determine an authenticity of each of the plurality of reagent strips, and perform a predetermined action when the authenticity of one or more of the plurality of reagent strips cannot be determined.

In some embodiments, the reader of the reagent strip analyzer may be an imager.

In some embodiments, the authentication code may further comprise a unique identifier value.

In some embodiments, the reagent strip analyzer may further comprise an input device, a communication device, or a combination thereof.

In some embodiments, the predetermined action causes the processor to output a notification to an output device, restrict further operation of the reagent strip analyzer, or a combination thereof.

According to some possible implementations, a method of authenticating a reagent strip may include illuminating a storage unit of the reagent strip with an optical signal in a non-visible spectrum; reading a non-visible code encoded in the storage unit of the reagent strip, the non-visible code encoded on the reagent strip with a substance only visible when illuminated by the optical signal in the non-visible spectrum; storing the non-visible code in a non-transitory computer readable medium; decoding the non-visible code using a truth table stored in the non-transitory computer readable medium to determine an authenticity of the urine reagent strip; and performing a predetermined action when the authenticity of the urine reagent strip cannot be determined. In some embodiments, the non-visible code may be encoded using a multi-dot pattern scheme and the truth table is a mapping scheme that is used to decode the non-visible code.

According to some possible implementations, a reagent strip may include a substrate having a first side and a second side; at least one reagent pad positioned on the first side of the substrate; and a storage unit positioned on at least one of the first side and a second side of the substrate, the storage unit encoded with a non-visible code using a substance only visible when illuminated by an optical signal in a particular and known spectrum, the non-visible code including control information.

In some embodiments, the non-visible code of the reagent strip may include one or more of: a 1D barcode, a 2D barcode, a QR code, a multi-dot pattern, and a numeric string.

In some embodiments, the control information of the reagent strip may comprise a unique identifier value.

In some embodiments, the non-visible code of the reagent strip may further comprise calibration information.

In some embodiments, the non-visible code of the reagent strip may further comprise an expiration date of the reagent strip.

In some embodiments, the non-visible code may include a multi-dot pattern scheme and a non-transitory computer readable medium of an analyzer may store a mapping scheme that a processor uses to decode the non-visible code to extract the control information.

### BRIEF DESCRIPTION OF THE DRAWINGS

To assist those of ordinary skill in the relevant art in making and using the inventive concepts disclosed herein, reference is made to the appended drawings and schematics, which are not intended to be drawn to scale, and in which like reference numerals are intended to refer to the same or similar elements for consistency. For purposes of clarity, not every component may be labeled in every drawing. Certain features and certain views of the figures may be shown exaggerated and not to scale or in schematic in the interest of clarity and conciseness. In the drawings:

FIG. 1 is a perspective view of an exemplary embodiment of a urine reagent strip system according to the inventive concepts disclosed herein.

FIG. 2 is a diagrammatic view of an exemplary embodiment of the urine reagent strip system of FIG. 1.

FIG. 3 is a front, plan view of a consumable having an authentication code stored on a storage unit in accordance with the inventive concepts disclosed herein.

FIG. 4 is a front, plan view of a plurality of consumables in a set of consumables having an authentication code stored on a storage unit in accordance with the inventive concepts disclosed herein.

FIG. 5a is a diagrammatic view of a reader of the urine reagent strip system of FIG. 1 illustrating an ultra-violet (UV) optical emission from an illumination source illuminating an ultra-violet spot of the storage unit.

FIG. 5b is a diagrammatic view of the reader of the urine reagent strip system of FIG. 1 illustrating the reading of an authentication code of the urine reagent strip.

FIG. 6 is a diagram showing the steps of an exemplary embodiment of a urine reagent strip authentication method according to the inventive concepts disclosed herein.

FIG. 7 is a front, plan view of a consumable having an authentication code encoded at various locations on a first side of the consumable in accordance with the inventive concepts disclosed herein.

### DETAILED DESCRIPTION

Before explaining at least one embodiment of the inventive concepts disclosed herein in detail, it is to be understood that the inventive concepts are not limited in their application to the details of construction and the arrangement of the components or steps or methodologies set forth in the following description or illustrated in the drawings. The inventive concepts disclosed herein are capable of other embodiments or of being practiced or carried out in various ways. Also, it is to be understood that the phraseology and terminology employed herein is for the purpose of description and should not be regarded as limiting the inventive concepts disclosed and claimed herein in any way.

In the following detailed description of embodiments of the inventive concepts, numerous specific details are set forth in order to provide a more thorough understanding of the inventive concepts. However, it will be apparent to one of ordinary skill in the art that the inventive concepts disclosed herein may be practiced without these specific details. In other instances, well-known features have not been described in detail to avoid unnecessarily complicating the instant disclosure.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherently present therein.

Unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concepts. This description should be read to include one or at least one and the singular also includes the plural unless it is obvious that it is meant otherwise.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example.

Further, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Finally, as used herein qualifiers such as “about,” “approximately,” and “substantially” are intended to signify that the item being qualified is not limited to the exact value specified, but includes some slight variations or deviations therefrom, caused by measuring error, manufacturing tolerances, stress exerted on various parts, wear and tear, and combinations thereof, for example.

As discussed above, there exists a need for a system and method for identifying and detecting unauthentic consumable products in a manner that does not alert potential counterfeiters to an instantly detectable authentication system. The prior art addressed the detection of counterfeit consumable products, but not in a manner that would allow an authentication system to go unnoticed by potential counterfeiters for a period of time after one of the counterfeit consumable products had been used, read by an analyzer, and results reported. The present disclosure describes a reagent strip system having a consumable with a non-visible code encoding control information that may form an authentication system. When the reagent strip system forms the authentication system, the control information includes an authentication code that is only visible when illuminated by an optical emission in a specific wavelength range of the light spectrum thereby concealing the authentication system from potential counterfeiters. The present disclosure will describe the consumable as a reagent strip by way of example. It should be understood, however, that the consumable can be a product other than a reagent strip, as discussed below.

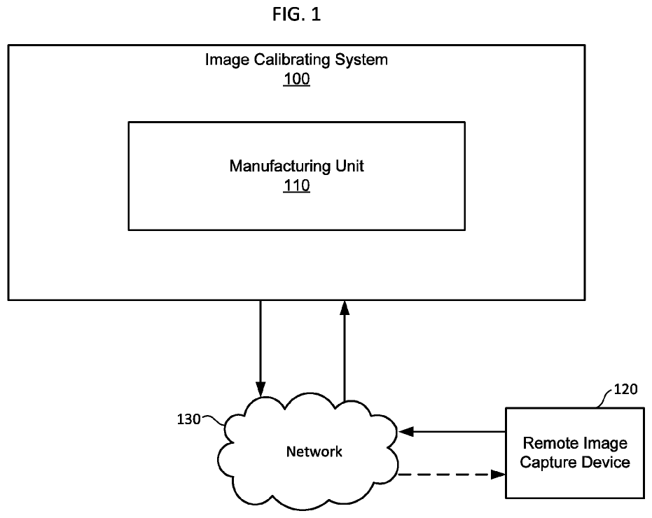

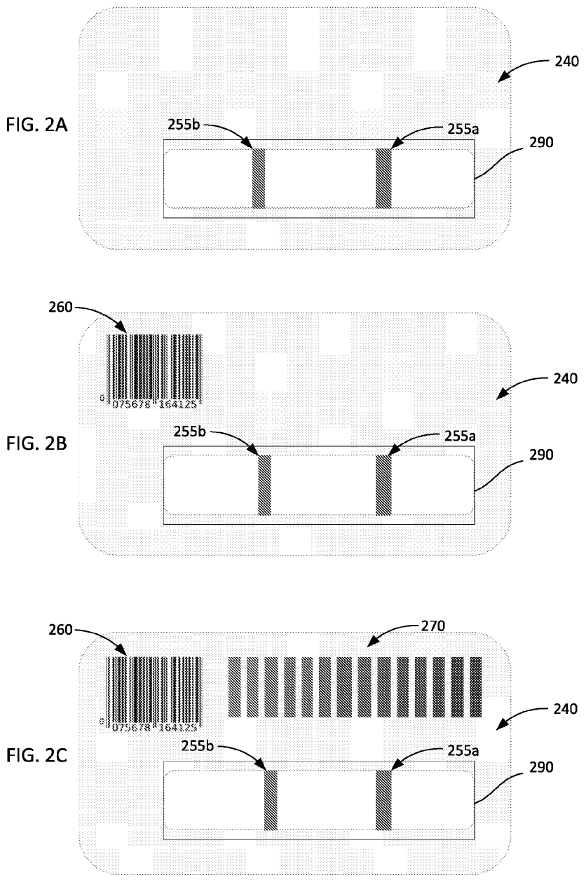

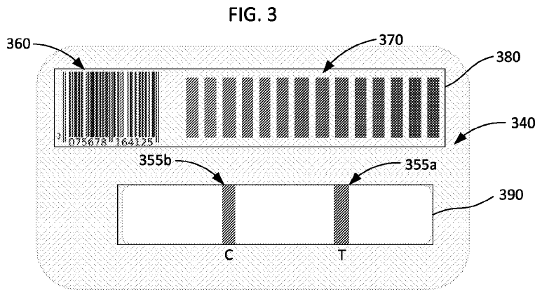

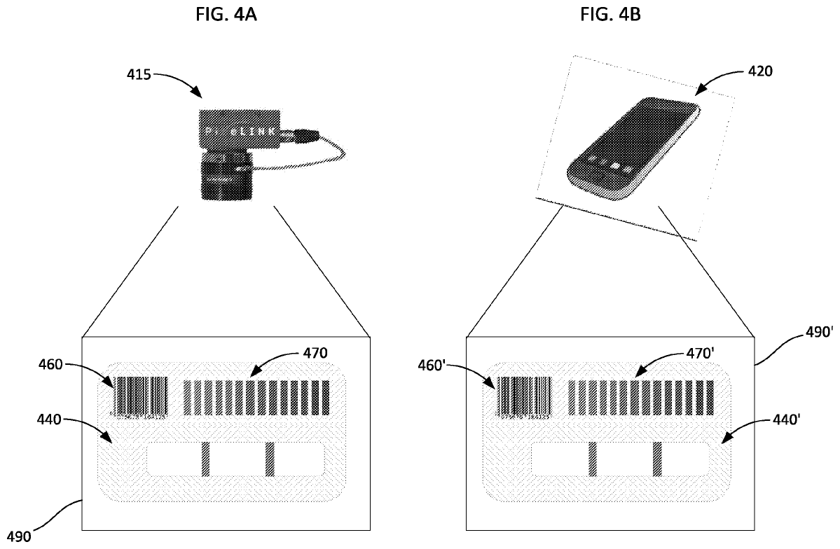

Referring now to FIGS. 1 and 2, shown therein is a perspective view of an exemplary embodiment of a reagent strip system 10 according to the inventive concepts disclosed herein and a diagrammatic view of the same, respectively. The reagent strip system 10 may be configured to have a first protection scheme 11 (shown in FIGS. 3 and 4) which determines authenticity of a reagent strip on a lot-by-lot basis.

As shown in FIG. 1, the reagent strip system 10 may include an analyzer 13. The analyzer 13 may be a reagent strip analyzer 14, for example. For the purposes of illustration, and not by way of limitations, the analyzer 13 will be described as a reagent strip analyzer 14, but it is to be understood that as used herein the term analyzer 13 is not limited to a reagent strip analyzer 14, but may also include point of sale systems, mobile cellular devices, inventory systems, pharmaceutical dispensary equipment, combinations thereof, and or the like, for example.

The reagent strip system 10 may also include a plurality of consumables 16, as shown in FIG. 2, although only one is shown in FIG. 1. The consumable 16 may be a reagent strip 18, for example. For the purposes of illustration, and not by way of limitation, the consumable 16 will be described as a reagent strip 18, but it is to be understood that as used herein the term consumable 16 is not limited to reagent strips 18, but may also include apparel and accessories, electronic good, toys, pharmaceuticals, food products, cosmetics, combinations thereof, and or the like, for example. The plurality of reagent strips 18 of the reagent strip system 10 will be discussed in further detail below with regards to FIGS. 3 and 4.

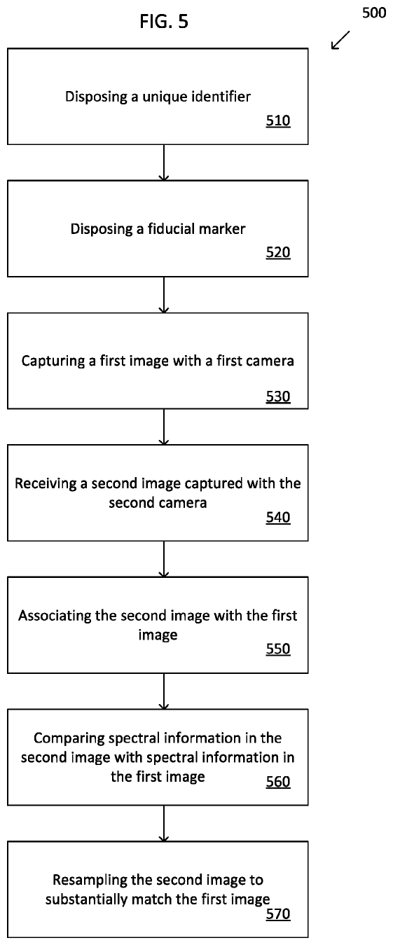

Generally, the reagent strip analyzer 14 may include a controller 20, a strip tray 24 configured to hold one or more reagent strip 18, and a reading system 28 with a housing 32. The housing 32 encloses a reading space 33 (see FIGS. 5A and 5B) sized and configured to receive the reagent strip 18. The housing 32 may be constructed of an opaque material to prevent light from outside the housing 32 from interfering with light being generated in the reading space 33 as discussed below. The housing 32 may be constructed from two or more optically opaque component(s) joined together so as to form and enclose the reading space 33. The reagent strip analyzer 14 may also include an optional waste ramp assembly and a waste receptacle (not shown), for example for disposing of the reagent strip 18 after the reagent strip 18 has been read by the reading system 28. The housing 32 may also be implemented to house and protect the various components of the reagent strip analyzer 14, and to protect technicians and laboratory work surfaces from contamination, for example. The reagent strip analyzer 14 may be provided with a strip feeder assembly 36 for moving the one or more reagent strip 18 from the strip tray 24 into the reading space 33 of the housing 32, past the reading system 28 to the waste receptacle. The strip feeder assembly 36 may include a conveyor, a set of drive wheels or the like. In some embodiments, the reagent strip analyzer 14 may not include a strip tray 24 and/or strip feeder assembly 36, it may, instead, include other features based on implementation. A specific implementation of the strip tray 24 and strip feeder assembly 36 is shown in FIGS. 1 and 2 as 24 and 36, respectively.

The controller 20 may include a processor 40 operably coupled with a non-transitory computer readable medium 44, a communication device 48, an input device 52, and an output device 56. The controller 20 may be operably coupled with the strip feeder assembly 36 and the reading system 28, for example.

The processor 40 may be implemented as a single processor or multiple processors working together or independently to execute processor executable code, such as application 60, implementing the logic described herein to authenticate the plurality of reagent strips 18, as will be described below. Embodiments of the processor 40 may include a digital signal processor (DSP), a central processing unit (CPU), a microprocessor, a multi-core processor, an application specific integrated circuit, a field programmable gate array, and combinations thereof, for example. The processor 40 may be at a location where the plurality of reagent strips 18 are being analyzed by the reagent strip analyzer 14, remotely, or combinations thereof. For example, the processor 40 may be contained within the housing 32, or remotely in a cloud-based computation service. In another embodiment, the processor 40 may include a first processor within the housing 32 and controlling the reading system 28 to obtain the readings from the plurality of reagent strips 18, and a second processor (not shown) receiving and analyzing the readings to determine the authenticity of the plurality of reagent strips 18.

The non-transitory computer readable medium 44 can be implemented as RAM, ROM, flash memory or the like, and may take the form of a magnetic device, optical device, or any other device configured to store processor executable instructions and information in a non-transitory manner, for example. The non-transitory computer readable medium 44 can be a single non-transitory computer readable memory, or multiple non-transitory computer readable memories functioning logically together or independently, for example. The application 60 can be stored in the non-transitory computer readable medium 44, read by the processor 40, and executed by the processor 40 to perform the logic described herein to authenticate the plurality of reagent strips 18 as will be described below.

The input device 52 may be capable of receiving information input from a user and/or processor 40, and transmitting such information to other components of the reagent strip analyzer 14. Implementations of the input device 52 may include, but are not limited to, a button, a keyboard, touchscreen, mouse, trackball, microphone, fingerprint reader, infrared port, slide-out keyboard, flip-out keyboard, cell phone, PDA, remote control, fax machine, wearable communication device, network interface, combinations thereof, and/or the like, for example.

The output device 56 may be capable of outputting information in a form perceivable by the user and/or processor 40. For example, implementations of the output device 56 may include, but are not limited to, a computer monitor, a screen, a touchscreen, a speaker, a website, a television set, a smart phone, a PDA, a cell phone, a fax machine, a printer, a laptop computer, combinations thereof, and the like, for example. It is to be understood that in some exemplary embodiments, the input device 52 and the output device 56 may be implemented as a single device, such as, for example, a touchscreen. It is to be further understood that as used herein the term user is not limited to a human being, and may comprise a computer, a server, a website, a processor, a network interface, a human, a user terminal, a virtual computer, combinations thereof, and/or the like, for example.

The strip tray 24 may be configured to accept the reagent strip 18, for example, and advance the reagent strip 18 so that the reagent strip 18 may be positioned in the reading space 33, and in a field of view of the reading system 28. While the strip tray 24 is illustrated as a retractable mechanism, it should be noted that the strip tray 24 may be implemented as a conveyor belt, a ratchet mechanism, a sliding ramp, or a strip-gripping or pulling mechanism, for example, configured to work with the strip feeder assembly 36 to advance the reagent strip 18 into the field of view of the reading system 28. In some exemplary embodiments, the strip tray 24 and/or the strip feeder assembly 36 may be operably coupled with the controller 20 and configured to move the reagent strip 18 (e.g., in an intermittent and stepwise manner) with a predetermined speed and time between each move so that each reagent strip 18 can be positioned across the field of view of the reading system 28 at known intervals of time, for example.

The reading system 28 may include a reader 64 and an illumination source 68, which may be fixed relative to the strip tray 24 and/or the strip feeder assembly 36, for example. In one embodiment, the reader 64 and the illumination source 68 are mounted to an inside surface of the housing 32.

The reader 64 may be implemented and function as any desired reader, and may be supported at a location above the strip tray 24 or other suitable location, so that a field of view of the reader 64 includes substantially the entire strip tray 24, for example. The reader 64 may be configured to detect, capture, read, image, or otherwise obtain, a signal indicative of the protection scheme 11 of the reagent strip system 10. The reader 64 may be an optical reader including, but not limited to including laser scanners, CCD scanners, or imager scanners. The reader 64 may also include any desired digital or analog imager, such as a digital camera, an analog camera, or a CMOS imager, a diode, and combinations thereof, for example. The reader 64 may also include a lens system, optical filters, collimators, diffusers, or any other optical-signal processing device, for example. Further, in some embodiments (not shown), the reading system 28 may include a first reader (not shown) which is an optical imager configured to detect an optical signal in the visible spectrum, and may further include a second reader (not shown) configured to detect an optical signal not in the visible spectrum such as a microwave imaging system, an X-ray imaging system, an ultra-violet imaging system, and other desired imaging systems, for example. The reader 64 may also be configured to transmit a signal indicative of the protection scheme 11 to the controller 20, for example. The signal indicative of the control scheme transmitted to the controller 20 by the reader 64 may be an electrical signal, an optical signal, and combinations thereof, for example.

The illumination source 68 may be located adjacent to the reader 64 as shown in FIG. 5A. The illumination source 68 may be implemented as one or more of a light emitting diode, a light bulb, a laser, an incandescent bulb or tube, a fluorescent light bulb or tube, a halogen light bulb or tube, or any other desired light source or object configured to emit an optical signal having any desired intensity, wavelength, frequency, or direction of propagation, for example. The illumination source 68 may be oriented such that substantially the entire field of view of the reader 64 is illuminated by the illumination source 68. In some embodiments, the illumination source 68 may be operably coupled with the controller 20 so that control and/or power signals may be supplied to the illumination source 68 by the controller 20. The optical signals emitted by the illumination source 68 may be conditioned or processed by one or more optical or other systems (not shown), such as filters, diffusers, polarizers, lenses, lens systems, collimators, and combinations thereof, for example. In some embodiments, more than one illumination source 68 may be implemented, such as a first illumination source (not shown) and second illumination source (not shown) having different locations and/or orientations. The first and second illumination sources may emit optical signals having different intensities and/or may be emitted for differing periods of time. In one embodiment, the reading system 28 may be operably coupled with the processor 40 so that one or more power and/or control signals may be transmitted to the reader 64 and/or to the illumination source 68 by the controller 20, and so that one or more signals may be transmitted from the reader 64 to the processor 40, for example.

The analyzer 13 may be connected to a network 70 that connects the analyzer 13 to an external system 72. As used herein, the terms “network-based,” “cloud-based,” and any variations thereof, are intended to include the provision of configurable computational resources on demand via interfacing with a computer and/or computer network, with software and/or data at least partially located on a computer and/or computer network.

In some embodiments, the network 70 may be the internet and/or almost any type of network. For example, in some embodiments, the network 70 may be a version of an internet network (e.g., exist in a TCP/IP-based network). It is conceivable that in the near future, embodiments within the present disclosure may use more advanced networking technologies.

In some embodiments, the external system 72 may optionally communicate with the analyzer 13 via the communication device 48 capable of interfacing with the network 70. For example, in one embodiment of the system 10, the external system 72 may supply data transmissions via the network 70 to the analyzer 13 regarding real-time or substantially real-time events (e.g., user updates, information updates, reagent strip information, calibration data, etc.). Data transmission may be through any type of communication including, but not limited to, audio or speech files, visual or image files, signals, textual, commands and/or the like. It should be noted that the external system 72 may be a server constructed and operating as is known in the art.

Referring now to FIGS. 3 and 4, shown therein is a front plan view of a consumable 16, and a plurality of consumables 16 in a set of consumables 115, respectively, in accordance with the inventive concepts disclosed herein. As discussed above, the consumables, may be reagent strips 18a-18c, e.g., reagent strips for analyzing urine.

As shown in FIG. 3, the reagent strip 18 may include a substrate 73, the protection scheme 11 positioned on, or otherwise associated with the substrate 73, and at least one reagent pad 86 positioned on, or otherwise associated with the substrate 73. Multiple reagent pads 86a-86j are shown by way of example.

The substrate 73 may be constructed of any suitable material, such as paper, photographic paper, polymers, fibrous materials, and combinations thereof, for example. The substrate 73 may have a first side 90 and an opposed second side 94 (see FIG. 5A). The protection scheme 11 and the reagent pads 86a-j can be located on the first side 90, the second side 94, or the protection scheme 11 may be located on one of the first side 90 and the second side 94 and the reagent pads 86a-j can be located on another one of the first side 90 and the second side 94.

The protection scheme 11 may include a storage unit 98 operable to store or encode information such as an authentication code, a manufacture date, an expiration date, a region of use, calibration parameters, what type of analytes are on the strip and how many there are, etc. The information stored or encoded in the storage unit 98 may be provided as a string of discreet numeric values. The information stored or encoded in the storage unit 98 may be stored on the storage unit 98 in the form of a 1D barcode, a 2D barcode, a QR code, a predetermined pattern or codes (e.g., spots, characters, symbols, dots or the like) and/or as a string of printed numbers that may be compared to a truth table or other mechanism.

The protection scheme 11 may be implemented as, for example, and not by way of limitation, as a non-visible code 111 or series of codes having a predetermined and known property such as being visible to the reader 64 when illuminated by the illumination source 68 emitting a specific wavelength or range of wavelengths of light. The specific wavelengths of light may be outside of a spectrum of light that is visible to a human. For example, the specific wavelengths of light may be in the infrared or ultraviolet spectrums of light. In one embodiment, the protection scheme 11 can be read by the same reader that detects the reagent pads, and at the same time.

In an exemplary embodiment, the non-visible code 111 may be implemented as a steganographic encoding system such as a multi-dot pattern character encoding scheme. In some embodiments, the multi-dot pattern may be a 16-dot pattern. In such an embodiment, even if a counterfeiter were to identify the specific wavelength of light to reveal the multi-dot pattern, to retrieve the information encoded therein the counterfeiter would still need to know the specific mapping scheme used to encode the characters with dot pattern. The specific mapping scheme may be generated and stored on the memory 44 of the reagent strip analyzer 14 or sent to the reagent strip analyzer 14 via the communication device 48.

The multi-dot pattern may be printed on the substrate 73 of the reagent strip 18 using a printer. The size of the dots in the multi-dot pattern may be calculated based on the size of the reagent strip 18 and the dots-per-inch (DPI) capability of a printer used to print the multi-dot pattern on the reagent strip 18, for instance. For example, the dots in the pattern may each have a cross-sectional dimension (e.g., diameter for a round dot) of 0.1 mm to 1.25 mm. In some embodiments, the cross-sectional dimension of the dots in the pattern may be in a range from 0.3 mm to 0.7 mm and more preferably 0.5 mm. The dots can be a variety of shapes including geometric shapes and non-geometric shapes. Exemplary shapes of the dots include square, rectangular, or circular.

While the protection scheme 11 has been described as a non-visible code 111, it is to be understood that as used herein the protection scheme 11 is not limited to a non-visible code 111, but may also include photo luminescent phosphorus (PLP), holograms, radio frequency identification, overt or visible features, combinations thereof, and or the like, for example.

In some embodiments, the non-visible code 111 may be covered or sealed to protect the infrared compound from a liquid sample as the liquid sample is being tested. The cover or seal may be formed of any material capable of preventing liquid from contaminating the infrared compound while remaining substantially translucent so that light from the illumination source 68 can pass through the cover or seal, and optical emissions from the infrared compound can be observed.

The location of the protection scheme 11 on the substrate 73 may be fixed. In some embodiments, the protection scheme 11 may be separate from the reagent pads 86a-j. In another embodiment, the protection scheme 11 may be positioned on the substrate 73 a predetermined distance from the reagent pads 86a-j.

The reagent pads 86a-j may be arranged in a grid-like configuration on the substrate 73 so as to define the reagent strip 18, for example. In one embodiment, the reagent pads 86a-j are aligned along the longitudinal axis of the reagent strip 18. In an exemplary embodiment, the reagent pads 86a-j may include fluidic or microfluidic compartments (not shown). The reagent pads 86a-j may be spaced apart a distance from one another so that adjacent reagent pads 86a-j may be simultaneously positioned at separate read positions within the field of view of the reading system 28 (FIGS. 1 and 2), for example. The reagent strip 18 may be a multiple-profile reagent strip having multiple reagent pads 86a-j having different reagents. Further, in some exemplary embodiments, the reagent strip 18 may include one or more calibration chip or reference pad, which may have no reagent and may serve as a color reference, for example.

Each reagent pad 86a-j may include a reagent configured to undergo a color change in response to the presence of a target constituent such as a molecule, cell, or substance in a sample of a specimen deposited on the reagent pad 86a-j. The reagent pads 86a-j may be provided with different reagents for detecting the presence of different target constituents. Different reagents may cause one or more color change in response to the presence of a certain constituent in the sample, such as a certain type of analyte. The color developed by a reaction of a particular constituent with a particular reagent may define a characteristic discrete spectrum for absorption and/or reflectance of light for that particular constituent. The extent of color change of the reagent and the sample may depend on the amount of the target constituent present in the sample, for example.

The sample may be any bodily fluid, tissue, or any other chemical or biological sample, and combinations thereof, such as urine, saliva, or blood, for example. In one non-limiting example, the sample is urine and constitutes one or more target constituent such as bilirubin, ketones, glucose, or any other desired target constituent, for example. The presence and concentrations of these target constituent(s) in the urine may be determinable by an analysis of the color change, for example, undergone by the one or more reagent pad 86a-j at predetermined times after application of the sample to the reagent pad 86a-j and/or at predetermined read positions in the field of view of the reading system 28, for example.

Now referring to FIG. 4 in combination with FIG. 3, shown in FIG. 4 is a front elevational view of a plurality of reagent strips 18a-c in the set of reagent strips 115 having information stored on the storage unit 98 (see FIG. 3) in accordance with the inventive concepts disclosed herein. A lot value may be associated with a particular quantity or lot of consumables produced by a manufacturer. As such, the plurality of reagent strips 18a-c from a particular quantity or same manufacturing lot may constitute a set of reagent strips 115, wherein each of the plurality of reagent strips 18a-c in a set of reagent strips 115 share an identical lot value, as shown in FIG. 4. In such an instance, where the consumer may have combined different sets of reagent strips 115, with different expiration dates, for example, the non-visible code 111 on each reagent strip 18 would allow the analyzer 13 to accurately assess whether the reagent strip 18 being analyzed is viable.

Referring now to FIG. 5a, shown therein is a diagrammatic view of the reader 64 of the reagent strip system 10 of FIG. 1, illustrating an optical emission from the illumination source 68 that illuminates the non-visible code 111. In reaction to being excited by the optical emission from the illumination source 68, the non-visible code 111 becomes visible to the reader 64 which is configured to detect or capture one or more optical or other signals indicative of a pattern of the non-visible code 111 and reagent pads 86a-j at any desired read position, and to transmit a signal indicative of the non-visible code 111 and/or reagent pads 86a-j at each read position to the processor 40, for example. One or more optical signals having wavelengths indicative of the luminescence and/or reflectance value of the non-visible code 111 and/or reagent pads 86a-i may be detected by the reader 64 at each read position, for example. The signal transmitted to the processor 40 by the reader 64 may be an electrical signal, an optical signal, and combinations thereof, for example. In one embodiment, the signal is in the form of an image file having a matrix of pixels, with each pixel having a color code indicative of its luminescence and/or reflectance value. In an exemplary embodiment, the image file may have two or more predetermined regions of pixels, each predetermined region of pixels corresponding to a read position of one of the PLP spot 111 and/or the reagent pads 86a-j in the field of view of the reader 64.

The processor 40 may determine the pattern of the non-visible code 111 when activated and/or reflectance value or the color change of reagent pad 86a-j and/or the reagent strip 18 along with a sample (e.g., urine) disposed on the reagent pad 86a-j and/or reagent strip 18, based on signals detected by the reader 64, for example. The color and/or reflectance value or the color change of reagent pads 86a-j and/or reagent strip 18 may be determined based upon the relative magnitudes of the reflectance signals of various color components, for example, red, green, and blue reflectance component signals. For example, the color reagent pads 86a-j emissions may be translated into a standard color model, which typically includes three or four values or color components (e.g., RGB color model, including hue, saturation, and lightness (HLS) and hue, saturation, and value (HSV) representation of points and/or CMYK color model, or any other suitable color model) whose combination represents a particular color. In some embodiments the reader 64 may detect multiple optical signals, with each detected signal having one or more color components, such as a red component signal, a green component signal, and a blue component signal, for example, and each of the component signals may be transmitted to the processor 40 via the same or separate communication link, such as a data bus. In some exemplary embodiments, the reader 64 may detect a single optical signal at each read position, and the processor 40 may translate a signal received from the reader 64 into separate color component signals such as a red component signal, a green component signal, and a blue component signal, for example.

Referring now to FIG. 5b, shown therein is a diagrammatic view of the reader 64 of the reagent strip system 10 of FIG. 1, illustrating the reading of the non-visible code 111 of the reagent strip 18.

The reader 64 may be configured to read control information stored on the non-visible code 111 of the storage unit 98 of each of the plurality of reagent strips 18a-c, and to transmit a signal indicative of the control information to the processor 40, for example. The signal transmitted to the processor 40 by the reader 64 may be an electrical signal, an optical signal, and combinations thereof, for example.

Upon receiving the signal from the reader 64, the processor 40 may store the control information on the non-transitory computer readable medium 44 of the reagent strip system 10. The processor 40 may then analyze the control information to determine the encoded information using the specific mapping scheme stored on the non-transitory computer readable medium 44. The mapping scheme may link specific control information to specific subroutines to be executed by the processor 40. For example, the encoded control information may include an authentication code, a lot number, a manufacture date, an expiration date, a region of use, calibration parameters, what type of analytes are on the reagent strip 18 and how many there are.

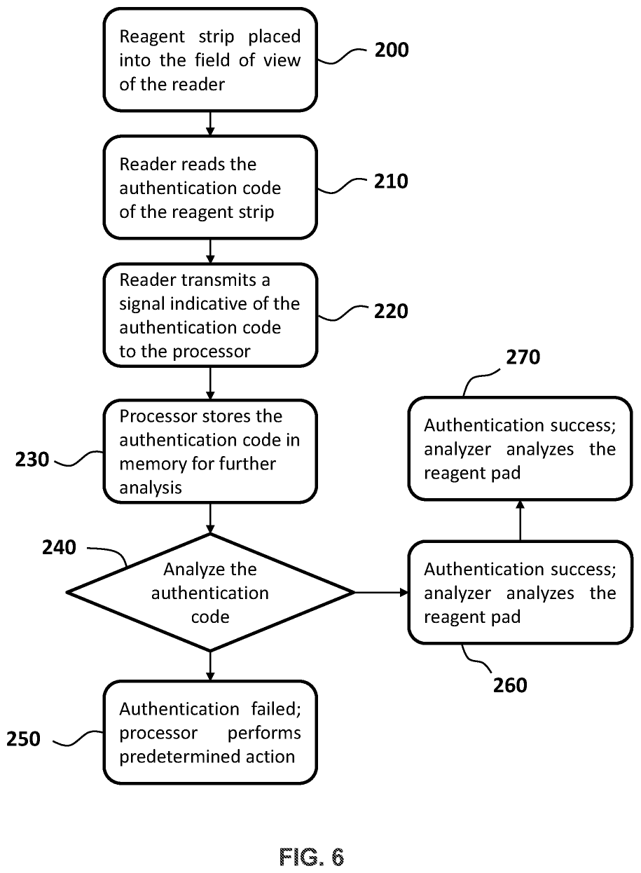

In one embodiment, the processor 40 authenticates the reagent strip using the authentication code. If the subroutine being executed by the processor 40 determines that the authentication code is not valid, the subroutine being executed by the processor 40 may inhibit further analysis of the reagent strip 18 or the set of reagent strips 115. This is due to the fact that, generally, reagent strips 18 are vulnerable to counterfeiting. When a counterfeit reagent strip 18 is used, readings from a reagent strip analyzer 14 may be prone to errors, and, thus, false readings may be obtained. Adding the protection scheme 11 having an authentication code security feature to a set of reagent strips 115 that is difficult to reproduce and capable of latent detection ensures that only authentic reagent strips 18 are analyzed. To that end, a consumable authentication method is shown in FIG. 6, which may be carried out by the processor 40 of the reagent strip system 10. In one embodiment, the authentication routine proceeds as set forth below, as shown in FIG. 6. In another embodiment, the position and orientation of the non-visible code 111 could relay to the processor 40 the position and orientation of the reagent strip 18, e.g., a proper alignment, or a misalignment relative to the reader 64.

The authentication routine will be described below, by way of example, as operating on a single reagent strip 18. The authentication routine can be repeated to operate on multiple reagent strips 18a-c either sequentially or simultaneously. In other words, the authentication routine can operate on the reagent strip 18a, followed by the reagent strip 18b, etc. Or, the authentication routine can utilize multiple readers 64 to simultaneously operate on multiples of the reagent strips 18a-c.

In step 200 the reagent strip 18 is placed into the field of view of the reader 64 of the reagent strip system 10 and positioned such that the authentication code stored on the storage unit 98 of the reagent strip 18 is within the field of view of the reader 64. In step 210, the reader 64 reads the authentication code of the reagent strip 18, and in step 220 the reader 64 transmits a signal indicative of the authentication code to the processor 40.

In step 230, the processor 40 stores the authentication code in the non-transitory computer readable medium 44 for further analysis.

In step 240, the processor 40 analyzes the authentication code to determine the authenticity of the reagent strip 18. In some embodiments, the processor 40 may further determine the lot number, manufacture date, expiration date, region of use, and/or calibration parameters, if present.

If the processor 40 determines that the authentication code fails to conform to the predetermined function, in step 250 the processor 40 performs a predetermined action. In one embodiment, the predetermined action may include the processor further restricting the reagent strip analyzer 14 from further analyzing the reagent strip 18 or the plurality of reagent strips 18a-c. In another embodiment, the predetermined action may include the processor 40 outputting a notification to a user on the output device 56.

If the processor 40 determines that the authentication code is valid, in step 260 the processor 40 will permit the reagent strip analyzer 14 to analyze the reagent pads 86a-j.

In step 270, the processor 40 further analyzes one or more of the reagent strips 18 as described above.

The consumable authentication method may be implemented as a set of processor executable instructions or logic stored on the non-transitory computer readable medium 44, that when executed by the processor 40, cause the processor 40 to carry out the logic to perform the steps as described above.

It is to be understood that the steps disclosed herein may be performed simultaneously or in any desired order. For example, one or more steps disclosed herein may be omitted. Further, one or more steps may be further divided into one or more sub steps, and two or more steps or sub-steps may be combined in a single step, for example. Further, in some exemplary embodiments, one or more steps may be repeated one or more times, whether such repetition is carried out sequentially or interspersed by other steps or sub-steps. Additionally, one or more step or sub-steps may be carried out before, after, or between, the steps disclosed herein, for example.

In addition to authentication information, the non-visible code 111 may be encoded with other control information such as, lot number, calibration information, manufacture date, and expiration date, for instance, that improve the accuracy of testing. For instance, when a reagent strip 18 is illuminated by the illumination source 68 and read by the reader 64, the application 60 may cause the processor 40 to decode the non-visible code 111 and determine the expiration date (control information) of the reagent strip 18. If the expiration date has passed, the subroutine selected by and being executed by the processor 40 of the reagent strip system 10 may cause the processor 40 to stop further processing and/or reading of the reagent strip 18 and notify the operator of the analyzer 13 that the expiration date has passed by displaying a notice, for instance, on the output device 56.

In another embodiment, the reagent strip system 10 may be programmed to determine an expiration date of the reagent strip 18 using the manufacture date. If the subroutine selected by and being executed by the processor 40 of the reagent strip system 10 determines that the reagent strip 18 has expired using the manufacture date, the analyzer 10 may be programmed to stop further reading and/or processing of the reagent strip 18 and notify the operator of the analyzer 13 that the reagent strip 18 has expired and should not be used by displaying a notice, for instance, on the output device 56.

The calibration information may, for instance, include chemical makeup of the reagents that make up the reagent pads 86a-86j of the test strip 18 and optimal testing conditions for those chemical makeups. In an exemplary embodiment, the reagent ingredients for leukocyte testing may include 0.4% w/w derivatized pyrrole amino acid ester; 0.2% w/w diazonium salt; 40.9% w/w buffer; and 58.5% w/w nonreactive ingredients and the reagent ingredients for urobilinogen may include 0.2% w/w p-diethylaminobenzaldehyde; and 99.8% w/w nonreactive ingredients. In such an embodiment, results for urobilinogen and leukocytes may be decreased at temperatures below 22° C. and increased at temperatures above 26° C. The subroutine selected by and being executed by the processor 40 of the system 10 may notify the operator of the analyzer 13 of the optimal testing conditions and/or be programmed to stop processing of the reagent strip 18 if the optimal testing conditions are not present when the reagent strip 18 is processed.

In some embodiments, the calibration information may be encoded in the storage unit 98 on each reagent strip 18. In other embodiments, the calibration information may be retrieved from the external system 72 via the network 70 based on the lot number or other identifier encoded in the storage unit 98.

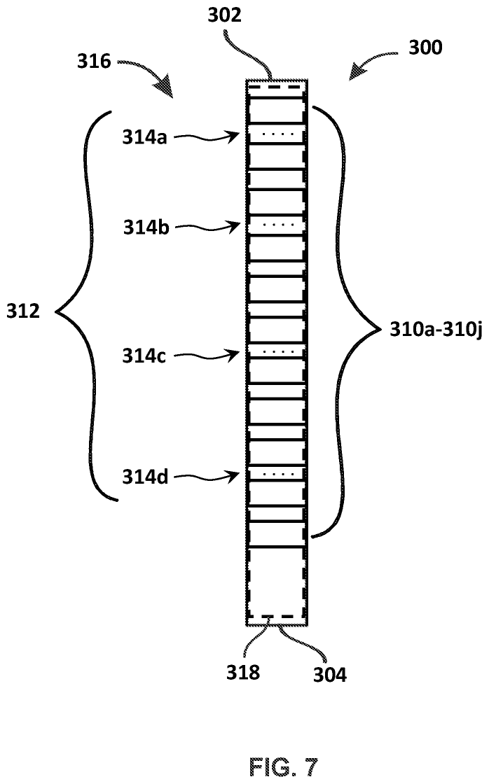

Referring now to FIG. 7, shown therein is a front plan view of a consumable 300 in accordance with the inventive concepts disclosed herein. As discussed above, the consumables, may be a reagent strip 302 which is similar to the reagent strip 18 described above. Therefore, in the interest of brevity, only the elements of reagent strip 302 that are different will be described in detail.

As shown in FIG. 7, the reagent strip 302 may include a substrate 304 having at least one reagent pad 310 positioned on or otherwise associated with the substrate 304. Multiple spaced apart reagent pads 310a-310j are shown by way of example. A protection scheme 312 may be positioned on or otherwise associated with the substrate 304. The protection scheme 312 may include a predetermined number of rows of characters 314 that may be positioned in predetermined positions on the substrate 304 such as between reagent pads 310a-310j. Four rows of characters 314a-314d are shown by way of example. In this embodiment, at least one row of characters 314 is positioned between adjacent reagent pads 310a-310j. Each row of characters 314 may be a predetermined number of characters. By way of example, the four rows of characters 314a-314d are shown each having four characters. The rows of characters 314a-314d together form a non-visible code 316. The non-visible code 316 may be positioned in a storage unit 318.

It should be noted that any number of rows of characters 314 having any number of characters may be used. While the rows of characters 314a-314d are shown positioned in between the reagent pads 310a-310j, in some embodiments, one or more of the rows of characters 314a-314d may be positioned anywhere within the storage unit 318, including on a second side of the substrate 304.

The following is a list of illustrative embodiments and is intended to complement, rather than displace or supersede, the previous descriptions.

1. A reagent strip system, comprising:
a reagent strip having a storage unit and a non-visible code encoded in the storage unit, the non-visible code encoded with a substance that produces a signal when illuminated by an optical signal in a particular and known spectrum;
an analyzer comprising:
an illumination device configured to project an optical signal in the particular and known spectrum onto at least a portion of the reagent strip;
a reader configured to capture an image of the signal produced by the non-visible code encoded in the storage unit of the reagent strip when illuminated by the optical signal from the illumination device;
a controller including a processor, and a non-transitory computer readable medium storing instruction that, when executed by the processor, cause the processor to:
extract control information from the non-visible code in the image of the signal produced by the non-visible code encoded in the storage unit; and
perform a predetermined action based upon the control information.

2. The reagent strip system of illustrative embodiment 1, wherein the control information further comprises a unique identifier value.

3. The reagent strip system of any one of illustrative embodiments 1-2, wherein the non-visible code includes one or more of: a 1D barcode, a 2D barcode, a QR code, a multi-dot pattern, and a numeric string.

4. The reagent strip system of any one of illustrative embodiments 1-3, wherein the analyzer is a reagent strip analyzer.

5. The reagent strip system of any one of illustrative embodiments 1-4, wherein the analyzer further comprises an input device, a communication device, or a combination thereof.

6. The reagent strip system of any one of illustrative embodiments 1-5, wherein the reader is an imager.

7. The reagent strip system of any one of illustrative embodiments 1-6, wherein the non-visible code comprises calibration information.

8. The reagent strip system of any one of illustrative embodiments 1-7, wherein the non-visible code further comprises an expiration date of the reagent strip.

9. The reagent strip system of any one of illustrative embodiments 1-8, wherein the predetermined action causes the processor to output a notification to an output device, restrict further operation of the analyzer, or a combination thereof.

10. The reagent strip system of any one of illustrative embodiments 1-9, wherein the non-visible code includes a multi-dot pattern scheme and the non-transitory computer readable medium further stores a mapping scheme that the processor uses to decode the non-visible code to extract the control information.

11. A reagent strip analyzer, comprising:
an illumination device configured to project an optical signal in a non-visible spectrum;
a reader operable to read a non-visible code from each of a plurality of reagent strips when illuminated by the optical signal from the illumination device, each of the plurality of reagent strips having a storage unit storing the non-visible code, the non-visible code encoded with a substance only visible when illuminated by the optical signal in the non-visible spectrum;
a controller including a processor and a non-transitory computer readable medium storing instructions that, when executed by the processor, cause the processor to:
store the information in the non-transitory computer readable medium;
analyze the information to determine an authenticity of each of the plurality of reagent strips; and
perform a predetermined action when the authenticity of one or more of the plurality of reagent strips cannot be determined.

12. The reagent strip analyzer of illustrative embodiment 11, wherein the reader is an imager.

13. The reagent strip analyzer of any one of illustrative embodiments 11-12, wherein the authentication code further comprises a unique identifier value.

17. The reagent strip analyzer of any one of illustrative embodiments 11-16, further comprising an input device, a communication device, or a combination thereof.

18. The reagent strip analyzer of any one of illustrative embodiments 11-17, wherein the predetermined action causes the processor to output a notification to an output device, restrict further operation of the reagent strip analyzer, or a combination thereof.

19. A method of authenticating a reagent strip, comprising:
Illuminating a storage unit of the reagent strip with an optical signal in a non-visible spectrum;
reading a non-visible code encoded in the storage unit of the reagent strip, the non-visible code encoded on the reagent strip with a substance only visible when illuminated by the optical signal in the non-visible spectrum;
storing the non-visible code in a non-transitory computer readable medium;
decoding the non-visible code using a truth table stored in the non-transitory computer readable medium to determine an authenticity of the urine reagent strip; and
performing a predetermined action when the authenticity of the urine reagent strip cannot be determined.

20. The method of illustrative embodiment 19, wherein the non-visible code is encoded using a multi-dot pattern scheme and the truth table is a mapping scheme that is used to decode the non-visible code.

21. A reagent strip, comprising:
a substrate having a first side and a second side;
at least one reagent pad positioned on the first side of the substrate; and
a storage unit positioned on at least one of the first side and the second side of the substrate, the storage unit encoded with a non-visible code using a substance only visible when illuminated by an optical signal in a particular and known spectrum, the non-visible code including control information.

22. The reagent strip of illustrative embodiment 21, wherein the non-visible code includes one or more of: a 1D barcode, a 2D barcode, a QR code, a multi-dot pattern, and a numeric string.

23. The reagent strip of any one of illustrative embodiments 21-22, wherein the control information comprises a unique identifier value.

24. The reagent strip of any one of illustrative embodiments 21-23, wherein the non-visible code further comprises calibration information.

25. The reagent strip of any one of illustrative embodiments 21-24, wherein the non-visible code further comprises an expiration date of the reagent strip.

26. The reagent strip system of any one of illustrative embodiments 21-25, wherein the non-visible code includes a multi-dot pattern scheme and a non-transitory computer readable medium of an analyzer stores a mapping scheme that a processor uses to decode the non-visible code to extract the control information.

From the above description, it is clear that the inventive concepts disclosed herein are well adapted to carry out the objects and to attain the advantages mentioned herein as well as those inherent in the inventive concepts disclosed herein. While exemplary embodiments of the inventive concepts disclosed herein have been described for purposes of this disclosure, it will be understood that numerous changes may be made which will readily suggest themselves to those skilled in the art and which are accomplished within the scope of the inventive concepts disclosed and as defined in the appended claims.

1. A method comprising:
disposing a unique identifier on an object;
disposing a fiducial marker on the object, the fiducial marker including a plurality of color regions:
capturing a first image of the object with a first camera;
receiving a second image of the object, the second image captured with a second camera:
associating the second image with the first image based on the unique identifier:
comparing spectral information of at least a portion of the fiducial marker in the second image with spectral information of at least the portion of the fiducial marker in the first image; and
resampling the second image to substantially match at least the portion of the fiducial marker in the second image to at least the portion of the fiducial marker in the first image based on the comparison.

2. The method of claim 1, further comprising:
storing the first image in a cloud based server system; and
referencing the first image in the cloud based server system with the unique identifier.

3. The method of claim 1, wherein disposing the fiducial marker includes printing the plurality of color regions on the object.

4. The method of claim 1, further comprising:
correcting the second image for any physical distortion; and
quantifying and correlating the second image with respect to the first image.

5. The method of claim 4, wherein correcting the second image includes applying via a cloud based server system, linear transformation of position vectors on the second image.

6. The method of claim 4, wherein linearizing the plurality of color regions includes applying via a cloud based server system, linear transformation on at least the plurality of color regions of the second image.

7. The method of claim 1, wherein the object is a diagnostic assay.

8. The method of claim 7, further comprising:
analyzing the second image to collect quantifiable data from the second image, the quantifiable data relating to a test associated with the diagnostic assay.

9. The method of claim 8, wherein the analyzing includes storing in a cloud based server system, information relating to the test and correlating via the cloud based server system, spectral information of at least a portion of the resampled second image to corresponding stored information relating to the test.

10. The method of claim 9, wherein correlating includes applying a curve fitting formula on spectral information of the at least a portion of the resampled second image.

11. The method of claim 1, wherein the fiducial marker is an adhesive label including the plurality of color regions.

12. The method of claim 1, wherein the unique identifier is at least one of a barcode, a hologram, and magnetic ink characters.

13. The method of claim 1, wherein the first image is captured under controlled conditions.

14. The method of claim 1, wherein spectral information includes at least one of intensity, wavelength, spectral value, Doppler shift, and Zeeman splitting.

15. The method of claim 1, wherein resampling the second image includes at least one of color correction, white balancing, gray balancing, gamma correction, and affine image transformation.

16. A method for collecting reliable data relating to a test from a second image, the method comprising:
disposing a unique identifier on a diagnostic assay;
disposing a spectral fiducial on the diagnostic assay, the spectral fiducial including a plurality of color regions;
capturing a first image of the diagnostic assay with a first camera;
at a processor:
receiving the second image of the diagnostic assay, the second image captured with a second camera;
associating the second image with the first image based on the unique identifier;
comparing spectral information of at least a portion of the spectral fiducial in the second image with spectral information of at least the portion of the spectral fiducial in the first image;
resampling the second image to substantially match at least the portion of the spectral fiducial in the second image to at least the portion of the spectral fiducial in the first image based on the comparison; and
correlating spectral information of at least a portion of the resampled second image to corresponding information relating to the test to collect reliable data,
wherein the test is associated with the diagnostic assay.

17. The method of claim 16, wherein the diagnostic assay is a lateral flow immunochromatographic assay.

18. The method of claim 16, wherein the spectral fiducial is an adhesive label including the plurality of color regions.

19. The method of claim 17, wherein the lateral flow immunochromatographic assay includes a control area and a test area.

20. The method of claim 19, wherein the at least a portion of the resampled second image is the test area in the resampled second image.

21. The method of claim 17, wherein the lateral flow immunochromatographic assay is a medical diagnostic assay used for at least one of home testing, point of care testing, and laboratory testing.

22. The method of claim 16, wherein spectral information includes at least one of intensity, wavelength, spectral value, Doppler shift, and Zeeman splitting.

23. The method of claim 16, wherein resampling the second image includes at least one of color correction, white balancing, gray balancing, gamma correction, and affine image transformation.

24. The method of claim 16, further comprising:
correcting the second image for any physical distortion; and
quantifying and correlating the second image with respect to the first image.

25. The method of claim 24, wherein correcting the second image includes applying via the processor, linear transformation of position vectors on the second image.

26. The method of claim 24, wherein linearizing the plurality of color regions includes applying via the processor, linear transformation on at least the plurality of color regions of the second image.

27. The method of claim 16, wherein correlating includes applying a curve fitting formula on spectral information of the at least a portion of the resampled second image.

28. The method of claim 16, wherein the unique identifier is at least one of a barcode, a hologram, and magnetic ink characters.

29. The method of claim 16, wherein the first image is captured under controlled conditions.

30. The method of claim 16, further comprising:
storing the first image in a cloud based server database.

31. The method of claim 16, wherein the reliable data includes specification, label, and quantity of an analyte.

32. The method of claim 16, wherein the processor is a cloud based server processor.

33. A system comprising:
a server processor; and
a manufacturing unit in digital communication with the server processor and configured to:
dispose a unique identifier on an object;
dispose a spectral fiducial on the object, the spectral fiducial including a plurality of color regions;
capture a first image of the object with an image capturing device; and
transmit the first image to the server processor;
the server processor configured to:
receive a second image of the object from a remote image capturing device;
associate the second image with the first image based on the unique identifier;
compare spectral information of at least a portion of the spectral fiducial in the second image with spectral information of at least the portion of the spectral fiducial in the first image; and
resample the second image to substantially match at least the portion of the spectral fiducial in the second image to at least the portion of the spectral fiducial in the first image based on the comparison.

34. The system of claim 33, wherein the server processor is a cloud based server processor.

35. The system of claim 33, further comprising:
a second image capturing device configured to:
capture the second image of the object; and
transmit the second image to the server processor.

36. The system of claim 33, wherein the spectral fiducial includes a plurality of color regions on the object.

37. The system of claim 33, wherein the object is a diagnostic assay.
